(19) United States
(12) Patent Application Publication
(10) Pub. No.: US 2015/0241634 A1
(43) Pub. Date: Aug. 27, 2015

(54) DOUBLE-FLUX LIGHTING DEVICE INCLUDING MULTIPLE OPTICAL FIBRES, AND ASSOCIATED PEROPERATIVE PROBE

(71) Applicant: COMMISSARIAT A L'ENERGIE ATOMIQUE ET AUX ENERGIES ALTERNATIVES, PARIS (FR)

(72) Inventors: Jean-Guillaume Coutard, Grenoble (FR); Michel Berger, Claix (FR); Sylvain Gioux, Creon (FR)

(21) Appl. No.: 14/421,126

(22) PCT Filed: Aug. 22, 2013
(86) PCT No.: PCT/EP2013/067446

§ 371 Date: Feb. 11, 2015

(30) Foreign Application Priority Data
Aug. 24, 2012 (FR) 1257974

(51) Int. Cl. G02B 6/32; A61B 5/00; F21V 8/00

(52) U.S. Cl. CPC G02B 6/32; G02B 6/0008; A61B 5/0071; A61B 5/0086

(57) ABSTRACT

In the field of lighting devices comprising two fiber sources of light and a light transport comprising a plurality of second multimode optical fibers, such devices are designed to be installed in intra-operative optical probes requiring a perfectly uniform visible illumination and fluorescence illumination. A lighting device is provided which comprises a single lens disposed between the fiber sources and the input of the light transport and an optical diffuser. A lighting device is also provided which comprises an afocal system disposed between the fiber sources and the input of the light transport and an optical diffuser. An optical fiber coupler or a semi-reflecting plate provides the mixing of the two fiber sources.

The field of the invention is that of lighting devices comprising a light transport comprising a plurality of multimode optical fibers. This transport is also known by the term “bundle”.

These optical transports have many applications, notably in the field of instrument optics for biomedical applications. These applications require lighting effects having particular characteristics. The applications designed for oncological surgery guided by fluorescence may in particular be mentioned.

Based on the capacity to excite then to collect the fluorescence of a tracer previously injected into the patient, oncological surgery requires, aside from the development of tracers specific to cancers, the design of intra-operative probes intended to guide the surgeon.

These probes must have very specific characteristics. These instruments must deliver a light, onto the tissues, for excitation of the fluorophore compatible with a good detection sensitivity, together with a bright white light in order for the surgeon to have a good view of the tissues being observed. In addition, certain medical applications require a reduced size of the optical head. By way of example, the diameter of the optical head of an intra-operative probe must not exceed a few tens of millimeters, typically between 15 and 50 millimeters.

In view of the end use of this illumination, the safety of the light delivered for living tissues and the eyes is also an imperative. With a given optical intensity, the source points delivered by the bundle of optical fibers therefore need to be multiplied in order to reduce their power and hence their damage potential. The objective is that, at the output of the probe, the illumination belongs to the class “laser 1” which is the lowest intensity class of energy, according to the French Act N^{o} 2010-750 of the 2^{nd} Jul. 2010 relating to the protection of workers against the risks due to artificial optical radiation and according to the European Directive N^{o} 2006/25/EC of the 5^{th} Apr. 2006 relating to the minimum health & safety prescriptions relating to the exposure of workers to the risks due to physical agents (artificial optical radiation).

Several lighting devices using optical fibers have been provided. The lighting device described in the U.S. Pat. No. 4,272,156 will notably be mentioned, whose idea is to cut the output faces of the optical fibers according to particular profiles and inclinations in order to improve the uniformity of the illumination. The lighting device described in the U.S. Pat. No. 5,412,749 will also be mentioned which allows the light coming from two different sources to be mixed within a single optical fiber. Lastly, the device described in the U.S. Pat. No. 4,964,692 will be mentioned, which describes an optical system, disposed at the output of the “bundle”, which allows a uniform illumination to be obtained.

These devices do not allow all of the characteristics and the limitations of an intra-operative probe, such as have been defined hereinabove, to be taken into account. The multiple fiber-optic dual-beam lighting device according to the invention meets this objective. It comprises optical means configured in such a manner as to obtain at the input of the bundle an illumination of the same intensity and of the same aperture on each fiber so as to obtain a more uniform illumination at the output. ‘More uniform illumination’ is understood to mean that the light intensity delivered by each fiber has a low dispersion. On the other hand, this method allows the total light intensity provided by the illumination to be optimized without ever exceeding the permitted safety levels on each optical fiber. Indeed, when the illumination is non-uniform, if only one optical fiber is above the permitted threshold, then the lighting device does not comply with ocular safety standards. In contrast, with the invention, owing to the uniformity of the light intensity delivered by each fiber, the light intensity from each fiber can be close to the maximum permitted light intensity. The illumination is therefore more uniform and more intense, while at the same time complying with the safety standards.

More precisely, the subject of the invention is a lighting device comprising at least one fiber light source and a light transport comprising a plurality of second multimode optical fibers, the light coming from the light source being guided to an output end of a first optical fiber, the first ends of the second optical fibers generally being grouped in a substantially uniform manner within a first surface, the second ends of the optical fibers being disposed around the periphery of a second surface, characterized in that the lighting device comprises:
a first lens disposed between the output end of the first optical fiber and the first ends of the second optical fibers, said output end of the first optical fiber being disposed substantially on the optical axis and at the primary object focal point of said first lens; and said first ends of the second optical fibers being disposed substantially on the optical axis and at the primary image focal point of said first lens;
an optical diffuser taking the form of a plate, in particular a thin plate, disposed between the first lens and the first ends of the second optical fibers.

Advantageously, the focal length of the first lens is substantially equal to the quotient of the radius of the first circular surface over the numerical aperture of the first optical fiber, and the scattering angle of the optical diffuser is substantially equal to the numerical aperture of the second optical fibers.

The first surface may be circular. The second surface is preferably annular, which allows a good adaptation around a camera of cylindrical shape. Preferably, the second ends of the optical fibers are disposed so as to be substantially equidistant around the periphery of said second surface.

The invention also relates to a lighting device comprising at least one fiber light source and a light transport comprising a plurality of second multimode optical fibers, the light coming from the light source being guided to an output end of a first optical fiber, the first ends of the second optical fibers generally being grouped in a substantially uniform manner within a first surface, the second ends of the optical fibers being disposed around the periphery of a second surface, characterized in that the lighting device comprises:
An afocal optical system comprising a first lens and a second lens, said system being disposed between the output end of the first optical fiber and the first ends of the second optical fibers, the primary image focal point of the first lens coinciding with the primary object focal point of the second lens;
The output end of the first optical fiber being disposed substantially on the optical axis and at the primary object focal point of said first lens; and said first ends of the second optical fibers being disposed substantially on the optical axis and at the primary image focal point of said second lens;
An optical diffuser taking the form of a plate disposed in the neighborhood of the primary image focal point of the first lens and at the primary object focal point of the second lens.

The first surface may be circular. The second surface is preferably annular, which allows a good adaptation around a camera of cylindrical shape. Preferably, the second ends of the optical fibers are disposed so as to be substantially equidistant around the periphery of said second surface.

The afocal system can have a unitary magnification and a numerical aperture substantially equal to the numerical aperture of the second optical fibers.

Generally speaking, the invention relates to a lighting device comprising at least one fiber light source and a light transport comprising a plurality of second multimode optical fibers, the light coming from the light source being guided to an output end of a first optical fiber, the first ends of the second optical fibers generally being grouped in a substantially uniform manner within a first surface, the second ends of the optical fibers being disposed around the periphery of a second surface, characterized in that the lighting device comprises:
A lens, disposed between the output end of the first optical fiber and the first ends of the second optical fibers, in such a manner that said first ends of the second optical fibers are disposed substantially at the primary image focal point of said lens;
An optical diffuser, taking the form of a plate composed of a diffusing material, disposed between the output end of the first optical fiber and the first ends of the second optical fibers.

According to one embodiment, the device comprises a first lens disposed between the output end of the first optical fiber and the first ends of the second optical fibers, said output end of the first optical fiber being disposed substantially on the optical axis and at the primary object focal point of said first lens, and said first ends of the second optical fibers being disposed substantially on the optical axis and at the primary image focal point of said first lens. According to this embodiment, the diffuser is preferably disposed in the neighborhood of the primary image focal point of the first lens.

According to another embodiment, the device comprises a first lens and a second lens, the primary image focal point of the first lens coinciding with the primary object focal point of the second lens, the output end of the first optical fiber then being disposed substantially on the optical axis and at the primary object focal point of said first lens, and said first ends of the second optical fibers being disposed substantially at the primary image focal point of said second lens. According to this embodiment, the diffuser is preferably disposed in the neighborhood of the primary image focal point of the first lens and at the primary object focal point of the second lens.

Advantageously, the lighting device comprises a second light source and an optical coupler comprising two inputs and one output, the second source having a second emission spectrum different from the first emission spectrum of the first source, the first light source being disposed in front of the first input of the coupler, the second light source being disposed in front of the second input of the coupler, the output of the coupler being disposed at the primary object focal point of the first lens.

Advantageously, the lighting device comprises a second fiber light source and a semi-reflecting plate,

the second source having a second emission spectrum different from the first emission spectrum of the first source, the light coming from the second light source being guided to an output end of a third optical fiber,

the semi-reflecting plate comprising a dichroic coating optimized in such a manner as to have a transmission maximum of the first emission spectrum and a reflection maximum of the second emission spectrum, the semi-reflecting plate being disposed such that the image of the output end of the third optical fiber, by reflection on the semi-reflecting plate, is superposed onto the image by transmission of the output end of the first optical fiber.

The invention also relates to an intra-operative probe comprising a camera and a lighting device such as defined hereinabove, the first spectrum of the first source being situated in the visible, the second spectrum of the second source being situated in a fluorescence spectrum situated in the near-infrared, the second ends of the optical fibers being disposed so as to be substantially equidistant around the periphery of the objective lens of the camera.

The invention will be better understood and other advantages will become apparent upon reading the description that follows, presented by way of non-limiting example and by virtue of appended figures amongst which:

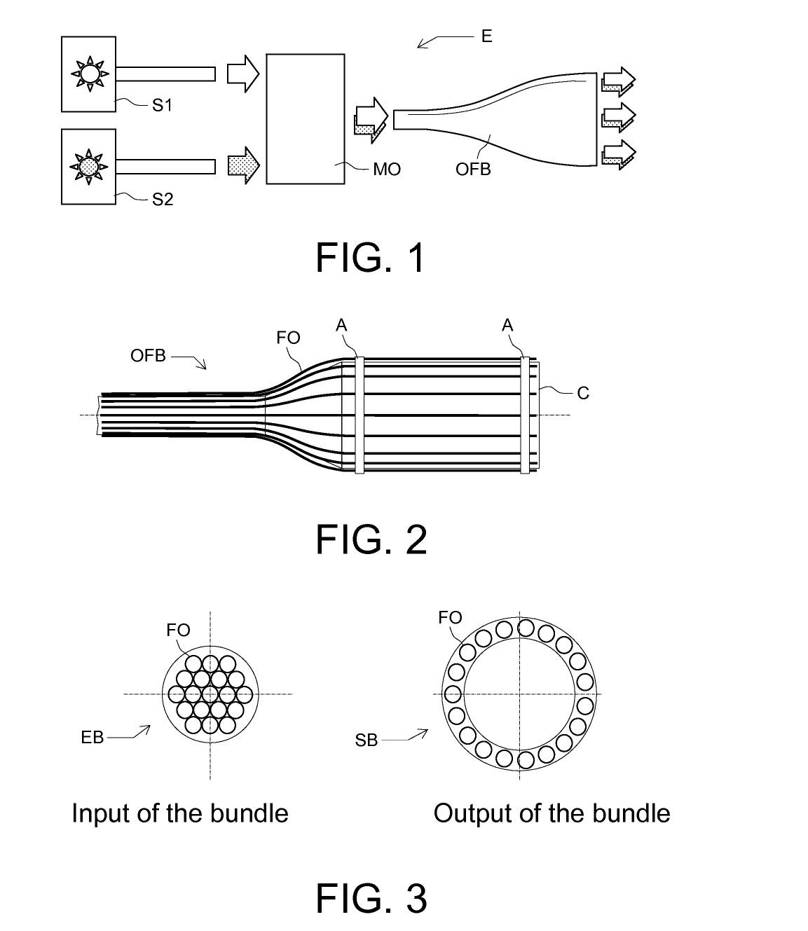

FIG. 1 shows an overall schematic diagram of a lighting device according to the invention;

FIGS. 2 and 3 show a view of the light transport of a lighting device according to the invention;

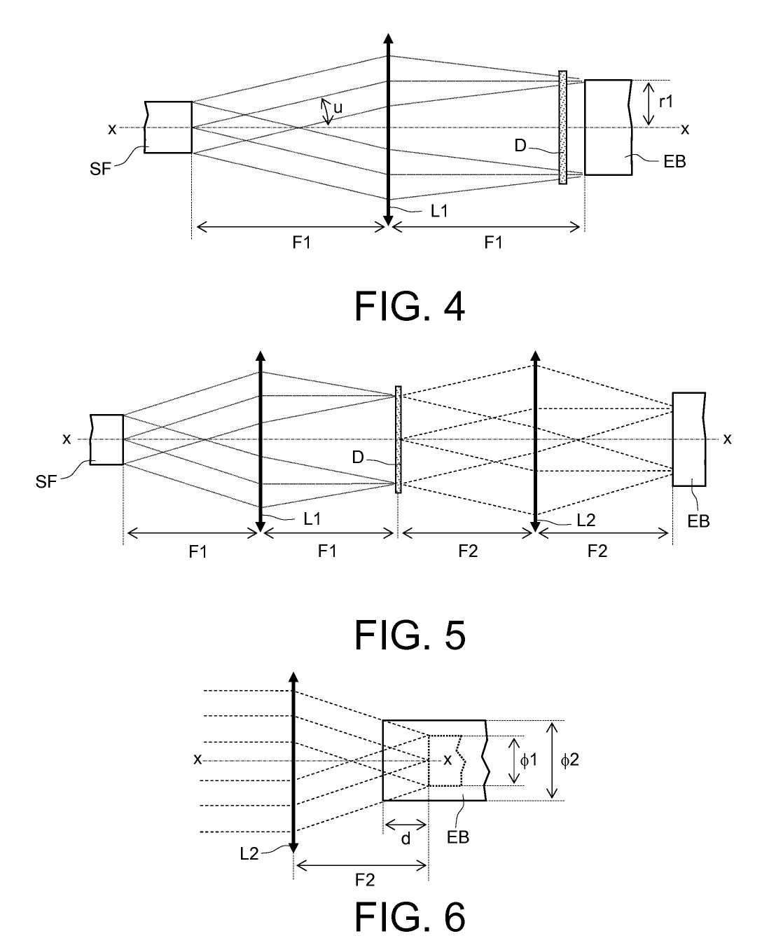

FIG. 4 shows a first embodiment of the optical interface means between the light source and the light transport;

FIGS. 5 and 6 show a second embodiment of the optical interface means between the light source and the light transport;

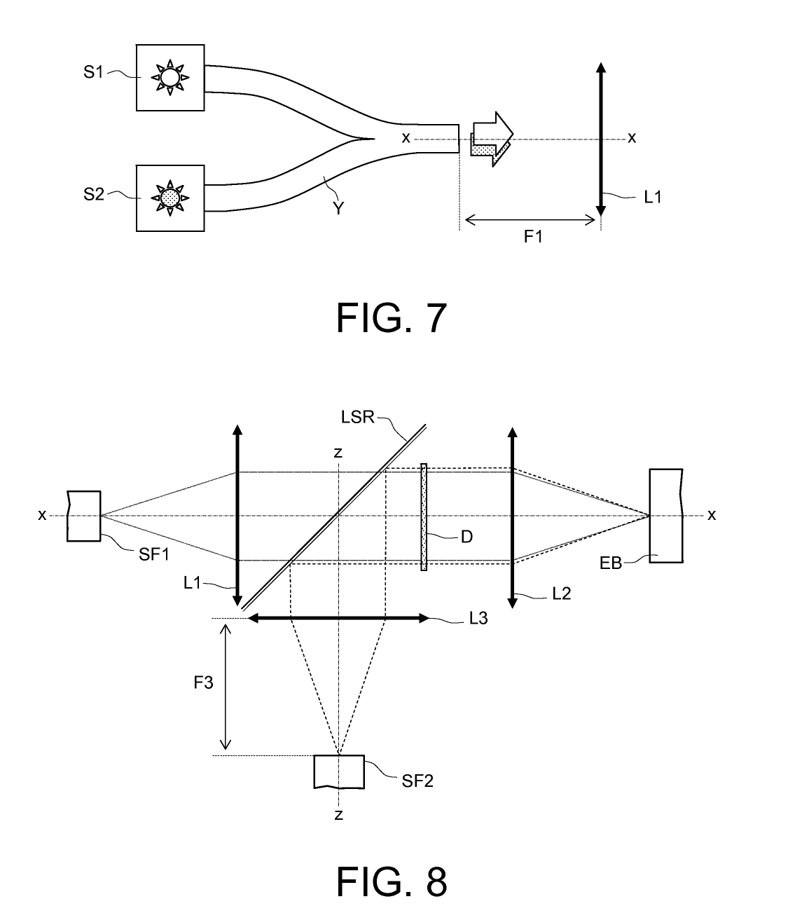

FIG. 7 shows a first means of coupling two different light sources;

FIG. 8 shows a second means of coupling two different light sources.

By way of example, FIG. 1 shows an overall schematic diagram of a lighting device E according to the invention. In this figure, it comprises two different fiber sources of illumination S1 and S2, optical means MO configured in such a manner as to interface the illuminating optical fiber or fibers with the input of a light transport OFB using multimode optical fibers FO or “optical fiber bundle”. The function of the light transport using optical fibers is two-fold. On the one hand, it allows the light to be carried and, on the other hand, it allows it to be distributed differently. FIGS. 2 and 3 illustrate these functions. FIG. 2 shows the output of the multimode optical fibers FO of the bundle. They are uniformly distributed around the optical head of a camera C that they surround. They are held around the optical head by two retaining rings A. FIG. 3 shows a front view of the input EB and a front view of the output SB of the bundle. At the input EB of the bundle, the ends of the optical fibers FO are grouped within a hexagon circumscribed within a first circular surface. At the output SB of the bundle, the ends of the optical fibers are disposed so as to be substantially equidistant around the periphery of a second circular surface. By way of example, nineteen multimode optical fibers are shown in FIG. 3.

This type of illumination is notably used in intra-operative optical probes. Generally speaking, as indicated in FIG. 1, the lighting device of this type of probe comprises two sources. The first is designed to provide an illumination in the visible spectrum, the second source, which is generally a laser source, emits in the near-infrared and is designed to cause a fluorescence radiation from the living tissues. The emission wavelength can be situated around 740 nanometers.

One of the difficulties of this type of illumination is the transmission of the light coming from the fiber light source at the input of the optical fibers of the optical bundle. Indeed, this light transmission must be effected with a minimum of losses. Each fiber of the bundle must receive substantially the same light intensity. Finally, the light beams entering into each fiber must have the same numerical aperture substantially corresponding to that of the optical fibers of the bundle. In the following, the numerical aperture of the fibers corresponds to the acceptance cone of the optical fibers conventionally defined by the following equation:

sin(i)=\sqrt{n₁²−n₂²}, i being the acceptance half-angle, n₁ being the optical index of the core of the optical fiber and n₂ being the optical index of the sheath of the optical fiber.

In order to best meet these requirements, the lighting device according to the invention comprises optical interfacing means. These means comprise one or two lenses and an optical diffuser.

FIGS. 4, 5, 6 and 8 show the interfacing means according to the invention. In these various figures, the optical lenses are represented conventionally by double arrows. These lenses may be thin lenses or optical means equivalent to thin lenses such as index-gradient lenses, for example. The light rays are represented by dashed lines.

FIG. 4 shows a first embodiment of these interfacing means. In this view, only one fiber source output SF is shown. As will be seen in FIG. 8, it is easy to adapt this device to the implementation of two fiber sources. The interfacing means comprise a first lens L1 disposed between the output end SF of the fiber source and the ends of the optical fibers of the input of the bundle EB, the output end SF of the fiber source being disposed substantially on the optical axis xx and at the primary object focal point of said first lens L1 and the first ends of the optical fibers of the input of the bundle being disposed substantially on the optical axis xx and at the primary image focal point of the first lens L1. An optical diffuser D taking the form of a thin plate is disposed between the first lens and the ends of the optical fibers of the bundle. The term ‘thin’ denotes the fact that its thickness is typically in the range between 1 and 20 mm. Placing the ends of the optical fibers of the input of the bundle EB at the focal distance from the first lens L1 allows the efficiency of the optical coupling to be optimized. It will be understood from FIG. 4 that, by placing the input of the bundle EB at a different distance, less than or greater than the focal distance, the amount of light collected by the bundle is reduced.

As indicated in FIG. 3, the ends of the optical fibers of the bundle are grouped in a substantially uniform manner in a first circular surface. Preferably, the focal length F1 of the first lens L1 is substantially equal to the quotient of the radius r1 of the first circular surface over the numerical aperture u of the optical fiber of the fiber source SF and the scattering angle α of the optical diffuser D is substantially equal to the numerical aperture u1 of the optical fibers of the bundle.

As can be seen in FIG. 4, all these dispositions offer the following advantages. Each point of the fiber source illuminates the totality of the first circular surface, the illumination thus being uniform over all the optical fibers of the input of the bundle. Each optical fiber of the bundle receives this illumination within an identical aperture angle. By virtue of the diffuser, this aperture angle corresponds perfectly to the numerical aperture of the optical fibers of the bundle, thus ensuring a perfectly uniform illumination of the optical fibers of the bundle.

FIG. 5 shows a second embodiment of these interfacing means. In this view, only one fiber source output is shown. As will be seen in FIG. 8, it is easy to adapt this device to the implementation of two fiber sources. The interfacing means comprise an afocal optical system comprising a first lens L1 and a second lens L2, said system being disposed between the output end of the fiber source SF and the ends of the optical fibers of the input of the bundle EB, the primary image focal point of the first lens L1 coinciding with the primary object focal point of the second lens L2. The output end of the fiber source SF is disposed substantially on the optical axis xx and at the primary object focal point of said first lens L1 and the first ends of the optical fibers of the input of the bundle are disposed substantially on the optical axis xx and at the primary image focal point of the second lens L2.

An optical diffuser D taking the form of a thin plate is disposed in the neighborhood of the primary image focal point of the first lens L1 and at the primary object focal point of the second lens L2, as can be seen in FIG. 5.

The afocal system can have a unitary magnification. Preferably, the numerical aperture of the lenses forming the afocal system is substantially equal to the numerical aperture of the optical fibers of the bundle. Furthermore, preferably, the numerical aperture of the optical fibers composing the bundle is substantially equal to the numerical aperture of the first optical fiber. When these three conditions are met, the focal distances F1 of the lens L1 and F2 of the lens L2 are equal. This disposition allows both a uniform illumination on the optical fibers of the bundle and an aperture angle of the illuminating beams perfectly adapted to the numerical aperture of the optical fibers of the bundle to be obtained. However, it is very easy to adapt this configuration to larger fiber surface areas or to different numerical apertures of the optical fibers by changing either the focal lengths of the lenses or the scattering angle of the diffuser.

As illustrated in FIG. 6, the illuminated surface of the bundle may be perfectly adapted by translating it over the optical axis of the afocal system. Thus, with a translation by a value of d, the system is able to go from an illuminated bundle surface area of diameter φ1 to an illuminated bundle surface area of diameter φ2. This distance d corresponds to an adjustment distance around the focal distance of the second lens L2. This adjustment distance depends on the numerical aperture of the optical fibers composing the bundle, on the diameter φ2 of the bundle and on the diameter φ1 of the first optical fiber. When the diameter φ2 is equal to the diameter φ1, in other words when the diameter of the first optical fiber is equal to the diameter of the input surface of the EB of the bundle, there is no adjustment to be made. The input of the bundle EB is disposed at the focal distance F2 from the lens L2. However, it is usual for the diameter φ2 of the bundle EB to be different and, in general, greater than the diameter φ1 of the first optical fiber. For example, φ2 can be equal to 2000 μm whereas φ1 is equal to 1500 μm.

In this case, the adjustment distance is obtained by the expression:

d=(φ2−φ1)/tan(α)

with ON=sin α, ON representing the numerical aperture of the optical fibers of the bundle.

Considering an aperture ON of 0.39, a diameter φ2 equal to 2000 μm and a diameter φ1 equal to 1500 μm, d is equal to 1180 μm. Thus, the adjustment distance is of the order of a millimeter.

It will then be understood that the input surface of the bundle is placed substantially at the focal distance F2 from the lens L2, in other words at the focal distance F2 potentially corrected by the adjustment distance d.

With respect to the first embodiment, this second embodiment, using an optical afocal system, is advantageous because it allows the bundle to be placed at a greater distance from the first optical fiber. This allows, for example, a dichroic plate to be more readily placed between the first optical fiber and the bundle, in the case where several sources of illumination are implemented.

Indeed, in a certain number of applications, it is necessary for the lighting device to comprise two different sources of illumination which are generally a white light source and an infrared source allowing a fluorescence light to be created. Various optical means allow the two sources to be coupled to the optical interfacing means in such a manner that the optical fibers of the bundle are simultaneously illuminated by the radiation coming from the two sources.

In a first embodiment shown in FIG. 7, the lighting device comprises an optical fiber coupler Y comprising two inputs and one output, the first light source S1 being disposed in front of the first input of the coupler, the second light source S2 being disposed in front of the second input of the coupler, the output of the coupler being disposed at the primary object focal point of the first lens. The coupler has two drawbacks. On the one hand, the standard couplers have input diameters of limited size. Generally speaking, the diameter does not exceed a millimeter. This small diameter can pose coupling problems in bundles comprising a large number of optical fibers and hence an input surface of significantly larger dimensions. The diameters of the optical fibers of the bundles are typically a few hundred microns. On the other hand, by its nature, the optical coupler has a transmission over each channel which does not exceed 60%.

Also, in a second embodiment shown in FIG. 8, the mixing of the two fiber sources is provided by a semi-reflecting plate LSR disposed at 45 degrees on the optical axis of the interfacing means after the lens L1. These interfacing means comprise a third lens L3 whose optical axis zz is disposed perpendicularly to the first optical axis xx and passes through the center of the semi-reflecting plate. This third lens has a focal length F3. The second fiber source SF2 is disposed at the focal point of this lens L3. The optical assembly composed of the lens L3, of the semi-reflecting plate LSR and of the lens L2 constitutes a second afocal system identical to the first. This disposition has two advantages over the previous one. On the one hand, it is easier to adapt the diameters of the fiber sources to that of the input of the bundle. On the other hand, it is possible to use a dichroic plate whose transmission and reflection coefficients are optimized so as to perfectly transmit or reflect the spectral bands of the two sources.

This embodiment, implementing two light sources, allows a single bundle to be used, where each fiber of the bundle may be illuminated either by the first light source or by the second light source, or by the two light sources simultaneously.

The lighting device according to the invention can be installed in various devices requiring a small, perfectly uniform, illumination. Thus, it can be used in intra-operative probes comprising a small camera. The second ends of the optical fibers of the bundle then surround the optical head of the camera. The illuminating ring has a thickness which does not exceed a millimeter and it can perfectly well comply with the conditions of ocular safety given that the laser illumination is distributed over a large number of optical fibers, each fiber delivering a uniform light intensity.

1. A lighting device comprising at least: one fiber light source; a light transport comprising a plurality of second multimode optical fibers, the light coming from the light source being guided to an output end of a first optical fiber, the first ends of the second optical fibers being grouped within a first surface, the second ends of the optical fibers being disposed around the periphery of a second surface;
an afocal optical system comprising a first lens and a second lens, said system being disposed between the output end of the first optical fiber and the first ends of the second optical fibers, the primary image focal point of the first lens coinciding with the primary object focal point of the second lens;
the output end of the first optical fiber being disposed substantially on the optical axis and at the primary object focal point of said first lens; and said first ends of the second optical fibers being disposed substantially on the optical axis and substantially at the primary image focal point of said second lens;
an optical diffuser taking the form of a plate disposed in the neighborhood of the primary image focal point of the first lens and at the primary object focal point of the second lens.

2. The lighting device as claimed in claim 1, wherein the afocal system has a unitary magnification.

3. The lighting device as claimed in claim 1, wherein the afocal system has a numerical aperture substantially equal to the numerical aperture of the second optical fibers.

4. The lighting device as claimed in claim 1, wherein the first ends of the second optical fibers are translated with respect to the primary image focal point of said second lens, through an adjustment distance determined as a function of the surface area of said first optical fiber and of the surface area of the first surface.

5. A lighting device comprising at least: one fiber light source; a light transport comprising a plurality of second multimode optical fibers, the light coming from the light source being guided to an output end of a first optical fiber, the first ends of the second optical fibers being grouped within a first surface, the second ends of the optical fibers being disposed around the periphery of a second surface,
a first lens disposed between the output end of the first optical fiber and the first ends of the second optical fibers, said output end of the first optical fiber being disposed substantially on the optical axis and at the primary object focal point of said first lens; and said first ends of the second optical fibers being disposed substantially on the optical axis and at the primary image focal point of said first lens;
an optical diffuser taking the form of a plate disposed between the first lens and the first ends of the second optical fibers.

6. The lighting device as claimed in claim 5, wherein the focal length of the first lens is substantially equal to the quotient of the radius of the first circular surface over the numerical aperture of the first optical fiber.

7. The lighting device as claimed in claim 5, wherein the scattering angle of the optical diffuser is substantially equal to the numerical aperture of the second optical fibers.

8. The lighting device as claimed in claim 1, wherein the lighting device comprises a second light source and an optical coupler comprising two inputs and one output, the second source having a second emission spectrum different from the first emission spectrum of the first source, the first light source being disposed in front of the first input of the coupler, the second light source being disposed in front of the second input of the coupler, the output of the coupler being disposed at the primary object focal point of the first lens.

9. The lighting device as claimed in claim 1, wherein the lighting device comprises a second fiber light source and a semi-reflecting plate,
the second source having a second emission spectrum different from the first emission spectrum of the first source, the light coming from the second light source being guided to an output end of a third optical fiber,
the semi-reflecting plate comprising a dichroic coating optimized in such a manner as to have a transmission maximum of the first emission spectrum and a reflection maximum of the second emission spectrum, the semi-reflecting plate being disposed such that the image of the output end of the third optical fiber, by reflection on the semi-reflecting plate, is superposed onto the image by transmission of the output end of the first optical fiber.

10. An intra-operative probe comprising a camera and a lighting device as claimed in claim 8, the first spectrum of the first source being situated in the visible, the second spectrum of the second source being situated in a fluorescence spectrum situated in the near-infrared, the second ends of the optical fibers being disposed so as to be substantially equidistant around the periphery of the objective lens of the camera.

11. An intra-operative probe comprising a camera and a lighting device as claimed in claim 9, the first spectrum of the first source being situated in the visible, the second spectrum of the second source being situated in a fluorescence spectrum situated in the near-infrared, the second ends of the optical fibers being disposed so as to be substantially equidistant around the periphery of the objective lens of the camera.

12. The lighting device as claimed in claim 5, wherein the lighting device comprises a second light source and an optical coupler comprising two inputs and one output, the second source having a second emission spectrum different from the first emission spectrum of the first source, the first light source being disposed in front of the first input of the coupler, the second light source being disposed in front of the second input of the coupler, the output of the coupler being disposed at the primary object focal point of the first lens.

13. The lighting device as claimed in claim 5, wherein the lighting device comprises a second fiber light source and a semi-reflecting plate,
the second source having a second emission spectrum different from the first emission spectrum of the first source, the light coming from the second light source being guided to an output end of a third optical fiber,
the semi-reflecting plate comprising a dichroic coating optimized in such a manner as to have a transmission maximum of the first emission spectrum and a reflection maximum of the second emission spectrum, the semi-reflecting plate being disposed such that the image of the output end of the third optical fiber, by reflection on the semi-reflecting plate, is superposed onto the image by transmission of the output end of the first optical fiber.

14. An intra-operative probe comprising a camera and a lighting device as claimed in claim 12, the first spectrum of the first source being situated in the visible, the second spectrum of the second source being situated in a fluorescence spectrum situated in the near-infrared, the second ends of the optical fibers being disposed so as to be substantially equidistant around the periphery of the objective lens of the camera.

15. An intra-operative probe comprising a camera and a lighting device as claimed in claim 13, the first spectrum of the first source being situated in the visible, the second spectrum of the second source being situated in a fluorescence spectrum situated in the near-infrared, the second ends of the optical fibers being disposed so as to be substantially equidistant around the periphery of the objective lens of the camera.
